(19) United States
(12) Patent Application Publication
(10) Pub. No.: US 2025/0335663 A1
(43) Pub. Date: Oct. 30, 2025

(54) CONTRASTIVE LEARNING FOR PEPTIDE BASED DEGRADER DESIGN AND USES THEREOF

(71) Applicant: UbiquiTx, New York, NY (US)

(72) Inventors: Kalyan PALEPU, New York, NY (US); Suhaas BHAT, New York, NY (US); Pranam CHATTERJEE, New York, NY (US)

(73) Assignee: UbiquiTx, New York, NY (US)

(21) Appl. No.: 18/868,481

(22) PCT Filed: May 23, 2023
(86) PCT No.: PCT/US2023/023255

§ 371 Date: Nov. 22, 2024

Related U.S. Application Data
(60) Provisional application No. 63/344,820, filed on May 23, 2022.

(51) Int. Cl. G06F 30/27

(52) U.S. Cl. CPC G06F 30/27

(57) ABSTRACT

A system and method of using contrastive language-image pre-training (CLIP) to devise a unified, sequence-based framework to design target-specific peptides via contrastive learning. In one or more further implementations, using known experimental binding proteins as scaffolds, a method is provided to generate a streamlined inference pipeline that efficiently selects peptides for downstream screening. In a further implementation, one or more compounds that are fused candidate peptides to E3 ubiquitin ligase domains that exhibit robust intracellular degradation of pathogenic protein targets in human cells.

### CROSS REFERENCE TO RELATED APPLICATION

The present application is National Stage Application of International Application No. PCT/US2023/023255, filed May 23, 2023, and claims priority to U.S. Patent Application No. 63/344,820, filed May 23, 2022, which are hereby incorporated by reference in their entireties. The International Application was published on Nov. 30, 2023 as International Publication No. WO 2023/230077 A1.

### FIELD OF THE INVENTION

The present disclosure relates to systems and methods contrastive language-image pre-training (CLIP) to devise a unified, sequence-based framework to design target-specific peptides via contrastive learning. Furthermore, by leveraging known experimental binding proteins as scaffolds, we create a streamlined inference pipeline, termed Cut&CLIP, that efficiently selects peptides for downstream screening. Finally, we experimentally fuse candidate peptides to E3 ubiquitin ligase domains and demonstrate robust intracellular degradation of pathogenic protein targets in human cells.

### BACKGROUND OF THE INVENTION

It has been estimated that while nearly 15% of human proteins are disease-associated, only 10% of such proteins interact with currently-approved small molecule drugs. Even more strikingly, of the 650,000 protein-protein interactions (PPIs) in the proteome, only 2% are considered “druggable” by pharmocological means Shin et al., 2020. Aside from small molecule-based approaches, monoclonal antibodies have found significant success in the clinic as biologics, but while they are highly selective and can bind antigens with high specificity, they are limited to extracellular PPIs and cannot naturally permeate the cell membrane Slastnikova et al., 2018. Peptides have been widely recognized as a more selective, effective, and safe method for targeting pathogenic proteins, due to their sequence-specific binding to regions of partner molecules Padhi et al., 2014, Buchwald et al., 2014. They have further demonstrated targeting of both extracellular and intracellular proteins, due to their small size and enhanced permeability, with or without conjugation to cell penetrating peptide (CPP) sequences Lindgren et al., 2000, Lozano et al., 2017, Adhikari et al., 2018.

Beyond standalone peptide binders, the inventors have created fused computationally-designed peptides to effector domains, such as E3 ubiquitin ligases, to enable binding and selective intracellular degradation of pathogenic targets of interest Chatterjee et al., 2020. Extending this “ubiquibody” (uAb) strategy to undruggable targets, including numerous oncogenic and viral proteins, represents a promising new therapeutic approach.

Current approaches for peptide engineering have relied on high-throughput screening and structure-based rational design, with the goal of redirecting to alternate targets, extending half-life in vivo, improving solubility, or preventing aggregation Fosgerau and Hoffmann, 2015. Experimental methods, such as large phage display libraries and quantitative binding assays, while effective at selecting strong candidate sequences, are laborious and expensive Wu et al., 2016, Kong et al., 2020, Carle et al., 2021. Structure-based methods for peptide design consist of interface predictors and peptide-protein docking softwares Raveh et al., 2011, Sedan et al., 2016, Tsaban et al., 2022. These approaches, however, rely heavily on the existence of co-crystal complexes consisting of the target protein, thus excluding disordered or unstable proteins, such as transcription factors, which have significant disease implications and are difficult to solve via experimental or computational protein structure determination methods Peterson et al., 2017, Das et al., 2018, Jumper et al., 2021.

Targeted protein degradation (TPD) has emerged as a promising approach to treat disease, but largely rely on small molecule warheads to bind to target proteins, excluding undruggable and disordered targets. As an alternative solution, our group designs ubiquibodies (uAbs), which are E3 ubiquitin ligase domains fused to a peptide specifically targeting a protein of interest. The design of these peptides, however, is quite challenging, and either requires high-throughput experimental screening or structure-based computational design, making unstructured and disordered targets particularly untenable.

Therefore, there is a need for the development of a sequence-based peptide generation platform, so as to rapidly and programmable design peptides to any target protein, especially those for which minimal structural information exists.

### SUMMARY OF THE INVENTION

A process for identifying binding peptides using a trained machine learning model, the process comprising: (1) Training a machine learning model to identify corresponding peptides to a target protein using a zero-shot transfer and multimodal learning algorithm; wherein the learning algorithm is jointly trained receptor and peptide encoders such that the cosine similarity between receptor embeddings and peptide embeddings; and (2) Utilizing the machine learning model to identify for a given target protein, at least one corresponding binding peptide. A process for identifying binding peptides using a trained machine learning model, the process comprising: (1) providing a target protein sequence to a trained machine learning model; and (2) generating at least one binding peptide sequence configured to bind to the target protein sequence.

The present disclosure relates to systems and methods contrastive language-image pre-training (CLIP) to devise a unified, sequence-based framework to design target-specific peptides via contrastive learning. Overall, our design strategy provides a generalized toolkit for designing peptides to any target protein without the reliance on stable and ordered tertiary structure, enabling generation of degraders to undruggable and disordered proteins such as transcription factors and fusion oncoproteins. Furthermore, by leveraging known experimental binding proteins as scaffolds, we create a streamlined inference pipeline, termed Cut&CLIP, that efficiently selects peptides for downstream screening.

Furthermore, a system and methods for evaluating candidate peptides by binding them to E3 ubiquitin ligase domains and demonstrating robust intracellular degradation of pathogenic protein targets in human cells is provided.

### FIGURES

FIG. 1 illustrates a flow diagram detailing the training process for one or more implementations of the machine learning models described herein.

FIG. 2 provides a chart detailing validation and testing of the trained model.

FIG. 3 illustrates a flow diagram of the peptide generation and ranking protocol described in one or more implementations.

FIG. 4 illustrates charts detailing the validation of the trained machine learning models described in one or more implementations herein.

FIG. 5 illustrates a flow diagram of an alternative peptide generation and ranking protocol described in one or more implementations.

FIG. 6 illustrates a validation of the trained machine learning models described in one or more implementations herein.

FIG. 7 illustrates one or more elements of the systems described.

FIG. 8 illustrates a flow diagram of one or more methods described.

### DETAILED DESCRIPTION

The text of any publications, materials, products referenced herein are hereby incorporated by reference as is provided in their respective entireties.

As used herein and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

As used herein and as well understood in the art, the term an “effective amount,” “sufficient amount” or “therapeutically effective amount” of an agent as used herein interchangeably, is that amount sufficient to effectuate beneficial or desired results, including preclinical and/or clinical results and, as such, an “effective amount” or its variants depends upon the context in which it is being applied. The response is in some embodiments preventative, in others therapeutic, and in others a combination thereof. The term “effective amount” also includes the amount of a compound of the disclosure, which is “therapeutically effective” and which avoids or substantially attenuates undesirable side effects.

As used herein and as well known in the art, and unless otherwise defined, the term “subject” means an animal, including but not limited a human, monkey, cow, horse, sheep, pig, chicken, turkey, quail, cat, dog, mouse, rat, rabbit, or guinea pig. In one embodiment, the subject is a mammal and in another embodiment the subject is a human patient.

As used herein, the term “homologous” refers to the subunit sequence similarity between two polymeric molecules, e.g., between two nucleic acid molecules, such as two DNA molecules or two RNA molecules, or between two protein molecules. When a subunit position in both of the two molecules is occupied by the same monomeric subunit; e.g., if a position in each of two DNA molecules is occupied by adenine, they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′-ATTGCC-5′ and 3′-TATGGC-5′ are 50% homologous. As used herein, “homology” is used synonymously with “identity.”

As used herein, the term “substantially the same” amino acid sequence is defined as a sequence with at least 70%, preferably at least about 80%, more preferably at least about 90%, even more preferably at least about 95%, and most preferably at least 99% homology to another amino acid sequence, as determined by the FASTA search method in accordance with Pearson & Lipman, Proc. Natl. Inst. Acad. Sci. USA 1988, 85:2444-2448.Therapeutic modalities targeting pathogenic proteins are the gold standard of treatment for multiple disease indications. Unfortunately, a significant portion of these proteins are considered “undruggable” by standard small molecule-based approaches, largely due to their disordered nature and instability. Designing functional peptides to undruggable targets, either as standalone binders or fusions to effector domains, thus presents a unique opportunity for therapeutic intervention.

By way of broad overview and introduction, the systems, methods and computer implemented processes described herein are directed to deep learning-based approaches to generating peptide binders that allow for customized protein degradation. As described in more detail herein, the inventors have developed a deep learning-based approach to generate the peptide binders used in ubiquibodies (“uAbs”) without the need or requirement of target structures. Such an approach represents a significant technical improvement in the field of computer derived binding sequences.

The described approach uses, in part, a neural network using the contrastive architecture. The inventors were able to use this neural network to predict specific peptide-protein binding.

As a further step, the inventors developed an inference pipeline, termed Cut&CLIP, which “cuts” likely candidate binding peptides as sub-sequences from known interacting partner sequences of the target protein, and then ranks them using the contrastive architecture based neural network. This approach reliably produces peptide-guided uAbs that induced degradation of several undruggable targets in vitro.

In a further arrangement, the presently pending systems, methods and computer implemented processes are directed to developing or generating binding peptides de novo. Rather than taking candidate peptide sequences from known interacting partners, the described approaches allow for the automatic generation of plausible binding peptide sequences using only a target protein sequence as an input. Here, the described generative process searches the latent space of a protein language model (“pLM”) such as the ESM-2 model.

More specifically, the described process or method samples from Gaussian distributions centered around the pLM (in one implementation the ESM-2) embeddings of naturally-occurring peptides and then decode those embeddings back to sequences. Where the pLM embedding space encodes expressive representations of protein sequences, the described process produces candidate peptides which are biochemically similar to naturally occurring peptides. Using a second model, referred to as the CLIP discriminator, the described process is able to screen these computationally generated peptides for binding activity to the target, and prioritize the top candidates for experimental testing.

In a further embodiment of the process for generating binding protein sequences, the systems, methods and computer implemented processes use a contrastive language-image pre-training (CLIP) to devise a unified, sequence-based framework to design target-specific peptides. In this implementation, known experimental binding proteins are used as scaffolds. Using these scaffolds a streamlined inference pipeline, termed Cut&CLIP, is used to efficiently selects peptides for downstream screening.

Once satisfactory experimentally candidates have been generated, they can be fused to E3 ubiquitin ligase domains in order to demonstrate robust intracellular degradation of pathogenic protein targets in human cells.

The inventors have found that the sequential structure of proteins, along with their hierarchical semantics, makes them a suitable target for language modeling. There exist language models that have been pre-trained on over 200 million natural protein sequences to generate latent embeddings that grasp relevant physicochemical, functional, and most notably, tertiary structural information. For example, see Rives et al., 2021, Elnaggar et al., 2020, Vig et al., 2020, Rao et al., 2020. Additionally, and perhaps even more interestingly, generative protein transformers have produced novel protein sequences with validated functional capability. See Madani et al., 2021. Through augmenting input sequences with their evolutionarily-related counterparts, in the form of multiple sequence alignments (MSAs), the predictive power of protein language models can be further strengthened. For example, see contact prediction results in Rao et al., 2021.

As described herein, the inventors have developed an approach to combine pre-trained protein language embeddings with novel contrastive learning architectures for the specific task of designing peptide sequences that bind target proteins and induce their degradation when fused to E3 ubiquitin ligase domains. By jointly training protein and peptide encoders to capture similarities between known peptide-protein pairs, the model described herein accurately evaluates peptide inputs as potential binders for embedded target proteins.

More specifically, to further downselect initial peptide candidate lists for queried targets, the systems, method and computer implemented processes described herein are directed to using predicted or experimentally-validated binding proteins as scaffolds for splicing, thus creating an integrated inference pipeline (referred to herein as as “Cut&CLIP”). As described in more detail herein, the Cut&CLIP method, as implemented by one or more processors or computers, reliably and efficiently generates peptides automatically, or otherwise without substantial human intervention. These generated peptides, when experimentally integrated within a uAb construct, are configured to induce robust degradation of pathogenic proteins in human cells.

Furthermore, the systems, methods and computer implemented processes described herein result in a more efficient and accurate approach to protein sequence generation compared to the existing art. Namely, in the past few years, protein structure prediction has experienced a wave of excitement with the advent of AlphaFold2. SeeJumper et al., 2021. With these prediction methods in hand, the protein design community has access to tools to generate custom proteins with enhanced or novel functionality. SeeAnishchenko et al., 2021, Cao et al., 2022.

However, the inventors have found that in some use-cases approaches using AlphaFold2 may be inferior to pure sequence-based models like the one described herein (referred to as the “Cut&CLIP” approach). Though the AF2-CoFold+PeptiDerive pipeline has been shown to produce viable protein degraders, this existing approach struggles to predict large and disordered protein complexes, highlighting its main drawback: efficiency. Thus, there is the need to provide an improved technical solution that is both more accurate and more efficient that existing approaches.

By way of example, in order to generate TRIM8-targeting peptides from PIAS3, the AF2-CoFold+PeptiDerive pipeline required 3 hours, 17 minutes, and 50 seconds on a powerful Amazon AWS p3.2xlarge instance with 8 CPU cores, 61 GB of RAM, and a Nvidia V100 GPU with 16 GB of VRAM, resources to which many researchers do not have access.

Cut&CLIP, on the other hand, only required 15 minutes and 58 seconds for the equivalent design task on a standard 2 CPU machine with 8 GB of memory. Thus, the present approach provides for a significant technological improvement in processing speed. Additionally, while both models produced highly effective peptides for TRIM8 and RBD, only Cut&CLIP produced effective degraders (>50% target degradation) for one of the most challenging cancer targets, KRAS. Therefore, the systems, methods and computer implemented processes described herein are directed to specific, identifiable technological solutions to existing technical problems found within the current state of the art.

To further contextualize the power of contrastive sequence-based models for protein design and screening, the model results shown here are based upon the strong assumption that within a batch of 250 peptides, only one is a viable binder. In most applications, especially when using a known interacting partner as a scaffold for peptide generation, there are likely multiple candidates that bind to the queried target. The experimental results support this observation, as potent degraders were identified by only testing 8 candidates for KRAS, RBD, and TRIM8. Overall, this work represents an approach for the application of sequence-based language models to therapeutically relevant protein design.

In one or more implementations of Cut&CLIP, for example, the described approach is configured to take advantage of powerful transformer architectures to better learn residue-residue interactions, will incorporate Kd values for high-affinity peptide design, and is leveraged to predict the off-targeting propensity of generated sequences. Most importantly, by integrating Cut&CLIP and uAb technology with effective delivery vehicles, such as adeno-associated vectors (AAVs) or lipid nanoparticles (LNPs), the peptide-guided protein degradation platform presented here serves as a one component of a therapeutic strategy to address a host of diseases deemed untreatable by standard small molecule-based means.

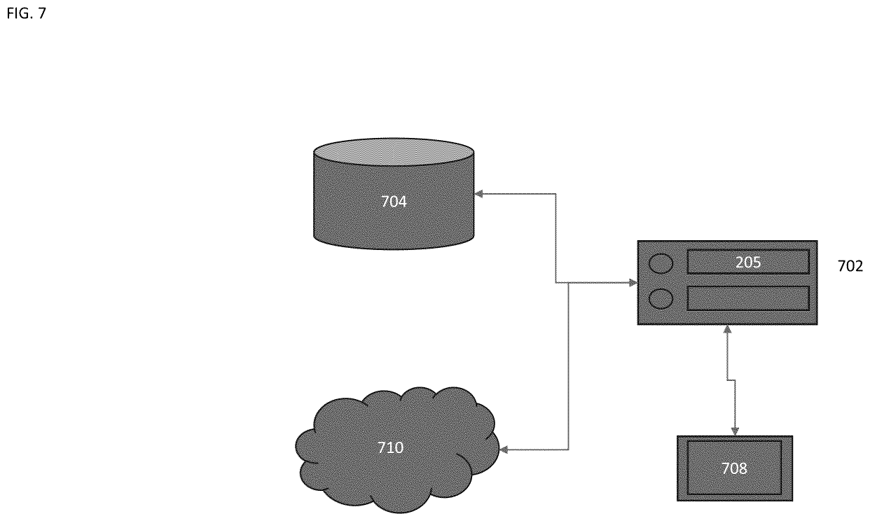

In one or more particular configurations, as shown in FIG. 7, the methods and processes described herein can be carried out by one or more processors or computers configured by code. For example, one or more processor(s) 702 are used to access data or data sets and evaluate them according to one or more functions provided for in one or more hardware or software modules. As used herein, the term “module” refers, generally, to one or more discrete components that contribute to the effectiveness of the presently described systems, methods and approaches. Modules can include software elements, including but not limited to functions, algorithms, classes and the like. In one arrangement, the software modules are stored as software in memory 205 of processor 702. Modules can, in some implementations, include discrete or specific hardware elements.

In one configuration, processor 702 is configured through one or more software modules to generate, calculate, process, output or otherwise manipulate the data obtained from a database 704.

In one implementation, processor 702 is a commercially available computing device. For example, processor 702 may be a collection of computers, servers, processors, cloud-based computing elements, micro-computing elements, computer-on-chip(s), home entertainment consoles, media players, set-top boxes, prototyping devices or “hobby” computing elements. Furthermore, processor 702 can comprise a single processor, multiple discrete processors, a multi-core processor, or other type of processor(s) known to those of skill in the art, depending on the particular embodiment. In a particular example, processor 702 executes software code on the hardware of a custom or commercially available cellphone, smartphone, notebook, workstation or desktop computer configured to receive data or measurements.

Processor 702 is configured to execute a commercially available or custom operating system, e.g., Microsoft WINDOWS, Apple OSX, UNIX or Linux based operating system in order to carry out instructions or code. In one or more implementations, processor 702 is further configured to access various peripheral devices and network interfaces. For instance, processor 702 is configured to communicate over the internet with one or more remote servers, computers, peripherals or other hardware using standard or custom communication protocols and settings (e.g., TCP/IP, etc.).

Processor 702 may include one or more memory storage devices (memories). The memory is a persistent or non-persistent storage device (such as an IC memory element) that is operative to store the operating system in addition to one or more software modules. In accordance with one or more embodiments, the memory comprises one or more volatile and non-volatile memories, such as Read Only Memory (“ROM”), Random Access Memory (“RAM”), Electrically Erasable Programmable Read-Only Memory (“EEPROM”), Phase Change Memory (“PCM”), Single In-line Memory (“SIMM”), Dual In-line Memory (“DIMM”) or other memory types. Such memories can be fixed or removable, as is known to those of ordinary skill in the art, such as through the use of removable media cards or modules. In one or more embodiments, the memory of processor 702 provides for the storage of application program and data files. One or more memories provide program code that processor 702 reads and executes upon receipt of a start, or initiation signal.

The computer memories may also comprise secondary computer memory, such as magnetic or optical disk drives or flash memory, that provide long term storage of data in a manner similar to a persistent memory device. In one or more embodiments, the memory of processor 702 provides for storage of an application program and data files when needed.

As shown in FIG. 7, processor 702 is configured to store data either locally in one or more memory devices. Alternatively, processor 702 is configured to store data, such as measurement data or processing results, in a local or remotely accessible database 704. The physical structure of database 704 may be embodied as solid-state memory (e.g., ROM), hard disk drive systems, RAID, disk arrays, storage area networks (“SAN”), network attached storage (“NAS”) and/or any other suitable system for storing computer data. In addition, database 704 may comprise caches, including database caches and/or web caches. Programmatically, database 704 may comprise flat-file data store, a relational database, an object-oriented database, a hybrid relational-object database, a key-value data store such as HADOOP or MONGODB, in addition to other systems for the structure and retrieval of data that are well known to those of skill in the art. Database 704 includes the necessary hardware and software to enable processor 108 to retrieve and store data within database 704.

In one implementation, each element provided in FIG. 7 is configured to communicate with one another through one or more direct connections, such as though a common bus. Alternatively, each element is configured to communicate with the others through network connections or interfaces, such as a local area network LAN or data cable connection. In an alternative implementation, processor 702 and database 704 are each connected to a network 710, such as the internet, and are configured to communicate and exchange data using commonly known and understood communication protocols.

In one arrangement, processor 702 communicates with a local or remote display device 708 to transmit, displaying or exchange data. In one arrangement, the display device 708 and processor 702 are incorporated into a single form factor, such as a sequencing device or other bioinformatics-based computing platform. In an alternative configuration, the display device 708 is a remote computing platform such as a smartphone or computer that is configured with software to receive data generated and accessed by processor 108. For example, processor 108 is configured to send and receive data and instructions from a processor(s) of a remote display device 708.

This remote display device 708 includes one or more display devices configured to display data obtained from processor 702. Furthermore, display device 708 is also configured to send instructions to processor 702. For example, where processor 702 and the display device are wirelessly linked using a wireless protocol, instructions can be entered into display device 708 that are executed by the processor 702. Display device 708 includes one or more associated input devices and/or hardware (not shown) that allow a user to access information, and to send commands and/or instructions to processor 702. In one or more implementations, the display device 708 can include a screen, monitor, display, LED, LCD or OLED panel, augmented or virtual reality interface or an electronic ink-based display device. Those possessing an ordinary level of skill in the requisite art will appreciate that additional features, such as power supplies, power sources, power management circuitry, control interfaces, relays, adaptors, and/or other elements used to supply power and interconnect electronic components and control activations are appreciated and understood to be incorporated.

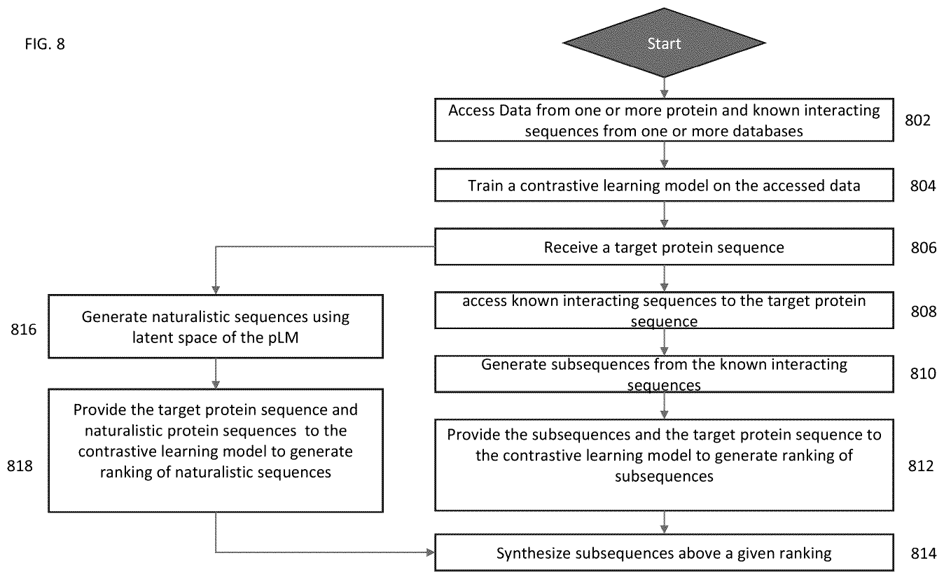

As shown in FIG. 8, a process for using the processor 702 to evaluate data and generate output information is provided. For example, one or more processors 702 are configured by code executing within a module to access protein sequence data from one or more remote databases 704. As shown in Step 802, data is accessed from protein databases for use in training a contrastive learning model.

As shown in step 804, the contrastive learning model is trained using accessed data. Once the model has been trained it can be stored in a database 704 for further use. Alternatively, once the contrastive learning model is generated, it can be used to generate potential peptide sequences to bind to a target protein.

For example, in step 806, a target protein is selected or entered into the working memory of the processor 702. The processor is then configured to select one or more known interacting sequences from a database 704, as shown in step 808. However, alternative databases or data storage devices can be used, including those data storage devices accessible via the internet via direct download, API, FTP, or another interface.

Once the known interacting sequences have been accessed, they are sliced into subsequences, as shown in step 810. These subsequences and the target protein sequence are provided to the trained contrastive learning model, which generates a ranking of each of the subsequences, as shown in step 812. Those subsequences having a value above a provided threshold are classified as having a high likelihood of binding to the target sequence. Those high-likelihood sequences are then provided for synthesis and experimental testing, as in step 814.

### Dataset Curation and Augmentation

It will be appreciated that the prior sequence generation systems have demonstrated utility using scaffold proteins to derive functional peptides for uAb generation. This is accomplished by executing the PeptiDerive protocol on co-crystals containing the target protein, thus identifying the linear polypeptide segments suggested to contribute most to binding energy. For example, see Chatterjee et al., 2020 and Sedan et al., 2016.

Therefore, in one or more implementations, a dataset of computationally derived presumptive peptides is generated according to a dataset generation step 802. For example, in one or more implementations, the PeptiDerive protocol is applied to complexes in the Database of Interacting Protein Structures (DIPS). See Sedan et al., 2016, Townshend et al., 2018. For example, in one or more implementations of the dataset generation step 502, the PeptiDerive protocol is run on every co-crystal in DIPS with a resolution of ≤2 Å, and the top 20mer peptide of each is selected to include in the dataset. By way of particular example, the following this process, a set of 28,517 peptide-receptor pairs can be generated.

In one or more further implementations, additional protein datasets can be combined to produce a larger data set. For example, in one or more implementations, an additional data set is added to the dataset generated using the PeptiDrive protocol. In one example, an additional dataset from Propedia, an experimentally-derived database that includes 19,814 peptide-receptor complexes from the Protein Data Bank (PDB). See Martins et al.

The protein sequences are clustered. For example, one or more clustering modules causes the protein sequences to be clustered at 50% sequence identity using MMSeq2. However, it will be appreciated that for specific applications or investigations, the percent sequence identity used for clustering can vary. For example, a range of sequence identity (from 10-90%) are understood and appreciated. Also see Steinegger and Söding. In one particular example, such clustering yielded, 7,434 clusters, and split the clusters into train, validation, and test splits according at a 0.7/0.15/0.15 ratio, respectively. However, it will be appreciated that alternative training, validation and test ratios are contemplated and understood.

In a further step, all sequences from the selected clusters were used in the train and validation splits, but only a single representative sequence for each cluster was employed for the test split, in order to ensure a reasonable balance of sequence diversity. Model Architecture and Training

One of the core problems in computer vision and NLP is model adaptation to new tasks and stress tests. However, there now exists a suitable architecture, termed CLIP (Contrastive Language-Image Pre-Training), which utilizes zero-shot transfer and multimodal learning to associate visual concepts in images and associate them with their names. For example, see Radford et al., 2021.

Without being held to any particular theory of implementation, the inventors have determined that just as CLIP connects images to their corresponding captions using jointly-trained image and caption encoders, CLIP-based architecture can be leveraged in a novel fashion to map target proteins to their corresponding peptides using jointly trained receptor and peptide encoders.

In one or more implementations, a training step is used to train the Clip architecture on the specific task indicated. For example, as shown in training step 506, encoders are trained such that the cosine similarity between receptor embeddings and peptide embeddings, defined as

$$k ( x , y ) = \frac{x \cdot y}{‖ x ‖ ‖ y ‖} \qquad ( 1 )$$

is near 1 for receptor-peptide pairs which do bind to each other, and is near −1 for receptor-peptide pairs which do not bind to each other. As input, the receptor encoder uses an MSA, while the peptide encoder simply uses the peptide sequence.

As part of the training step 804, the receptor and peptide encoders are trained on batches of n pairs of receptors and peptides which are known to interact. In one particular implementation, receptor MSAs and peptide sequences are encoded by their respective encoders, producing receptor embeddings r1, . . . , rn, and peptide embeddings p1, . . . , pn. The cosine similarity between all n2 receptor and peptide pairs is computed in a matrix K, defined as

$${K}_{ij} = k ( {r}_{i} , {p}_{j} ) \qquad ( 2 )$$

It is possible to interpret these cosine similarities as softmax logits. For instance, as part of the training step, logits were scaled by a learned temperature parameter t, which controls the model's degree of uncertainty in output probabilities, SeeHinton et al., 2015, and defines two sets of cross-entropy loss, one on matrix rows and one on its columns:

$${ℒ}_{r} = \frac{1}{n} {\sum }_{i = 1}^{n} log ( \frac{exp ( {K}_{ii} )}{{\sum }_{j = 1}^{n} exp ( {K}_{ij} )} ) \qquad ( 3 )$$

$${ℒ}_{p} = \frac{1}{n} {\sum }_{j = 1}^{n} log ( \frac{exp ( {K}_{jj} )}{{\sum }_{i = 1}^{n} exp ( {K}_{ij} )} ) \qquad ( 4 )$$

Here, Lr represents the loss on the model's ability to predict the correct receptor given a single peptide, while Lp represents the loss on the model's ability to predict the correct peptide given a single receptor.

By using these cross-entropy losses, we implicitly assumed that the n2-n receptor-peptide pairs in the batch which are not known interactions do not bind at all. While this may not be a completely accurate assumption, it is approximately true.

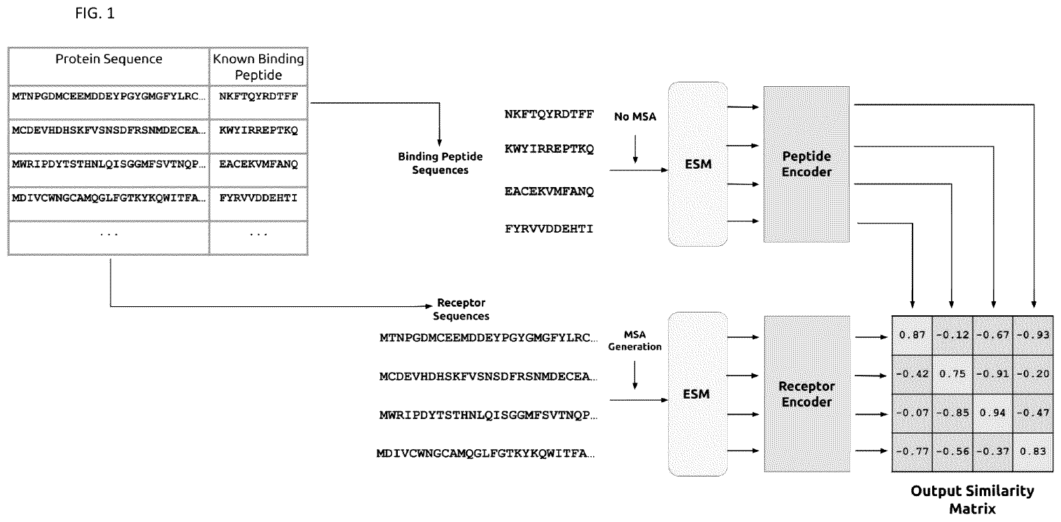

The model was then trained on the average of these two losses. The entire training process is illustrated in FIG. 1.

As shown in FIG. 1, the CLIP training process for peptide-protein pairs. Peptide and receptor encoders are jointly trained on ESM embeddings to predict high cosine similarities between known peptide-receptor embedding pairs and low cosine similarities for all other pairs.

In one particular implementation, receptor MSAs and peptide sequences were first input into the ESM pre-trained transformer protein language models introduced previously by Facebook. See Rives et al., 2021, Rao et al., 2020. These pre-trained models were trained on millions of diverse amino acid sequences, allowing the encoders to extract feature-rich embeddings, which are robust to sequence diversity while being trained on a relatively small dataset.

In one or more further implementations, the method or process described employed the ESM-MSA-1b model for the receptor MSAs, and ESM-1b for the peptide sequences, which does not require MSA inputs, as shown in FIG. 1. The receptor and peptide encoders were trained by taking these ESM embeddings as input. The receptor encoder and peptide encoder have identical architectures, though they differ in hyperparameters such as the number of layers.

Starting with an input I×ei ESM embedding (where I is the input sequence length and ei is the dimension of the ESM embedding), in one implementation h1 feedforward layers with ReLU activation on each amino acid embedding were applied separately, producing a I×eo embedding, where eo is the output embedding dimension produced by the encoder. Next, the embedding over the length dimension were averaged, producing an embedding vector of length eo. Then h2 feedforward layers with ReLU activation on the embedding vector are applied to obtain the output embedding.

As a relevant metric for model assessment in the context of screening, the top-k accuracy is calculated. This value represents the probability that the correct peptide is in the top k when provided a fixed batch of 250 candidate peptides, a suitable threshold for genetic screening. To calculate this metric, during prediction, the model is provided with a single protein target receptor and 250 peptides from the training set, one of which is a known binder. Over a batch of n receptor-peptide pairs, the mean reciprocal rank (MRR) is calculated.

Post-training, the derived final models demonstrate accurate ranking of known targeting peptides for a given target and vice versa, achieving 50% probability of identifying a correct candidate in the ranked top 50 out of 250, for example.

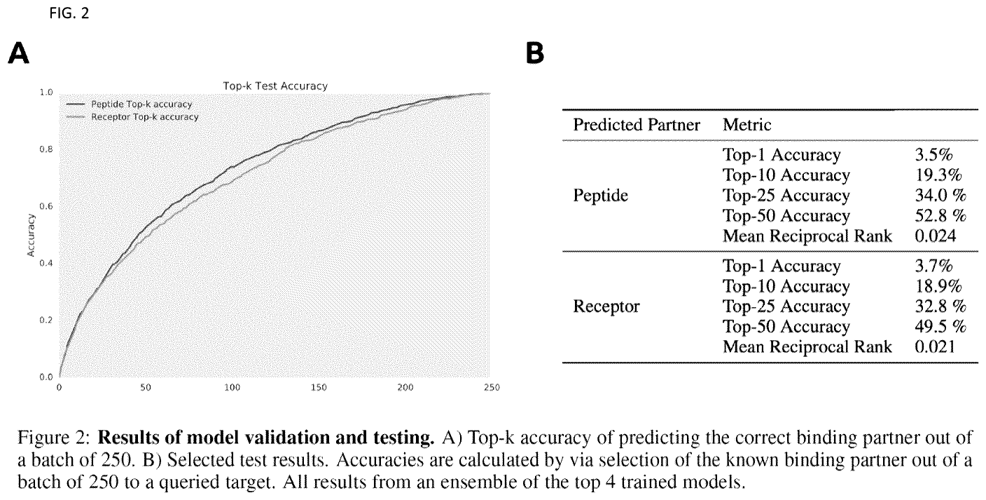

These results motivate not only model application for tractable peptide screening assays, but also its utilization to evaluate peptide specificity to a desired target, in comparison to off-target receptor proteins, as shown in FIG. 2.

It will be appreciated that the models described herein were trained on a single Nvidia V100 GPU with 32 GB VRAM, as well as 10 Xeon Gold 6248 CPU cores with a total of 90 GB of RAM. For model validation, over a batch of n receptor-peptide pairs, the mean reciprocal rank (MRR) was calculated as follows for both receptors and peptides, where ti is the rank of the known binding partner for the ith receptor-peptide pair:

$$M R R = \frac{1}{n} {\sum }_{i = 1}^{n} ( \frac{1}{{l}_{i}} ) \qquad ( 5 )$$

Top-k accuracy was calculated to be that the probability that the correct peptide is in the top k when provided a fixed batch of 250 candidate peptides. Peptide inference was conducted with a standard 2 CPU machine with 8 GB of RAM.

Turning now to FIG. 2, which provides the results of model validation and testing. FIG. 2A details the top-k accuracy of predicting the correct binding partner out of a batch of 250. FIG. 2B provides selected test results. Here, accuracies are calculated via selection of the known binding partner out of a batch of 250 to a queried target.

Once the model has been suitably trained, it can be employed to predict binding peptides using experimentally-validated interacting proteins for a queried target. It will be appreciated by those possessing an ordinary level of skill in the requisite art that unlike previous work using structural information, the current inference pipeline only requires the sequence of potential binders from established PPI databases or from experimental screening results. In turn, this allows for a system, method and computer implemented process that provides more flexible in identifying starting scaffolds. See Szklarczyk et al., 2020, Johnson et al., 2021. Specifically, the approach allows the computation of the CLIP peptide embedding for all k-mers of the interacting protein (where k is the desired size of the peptide), and rank them by their cosine similarities with the CLIP receptor embedding of the target protein.

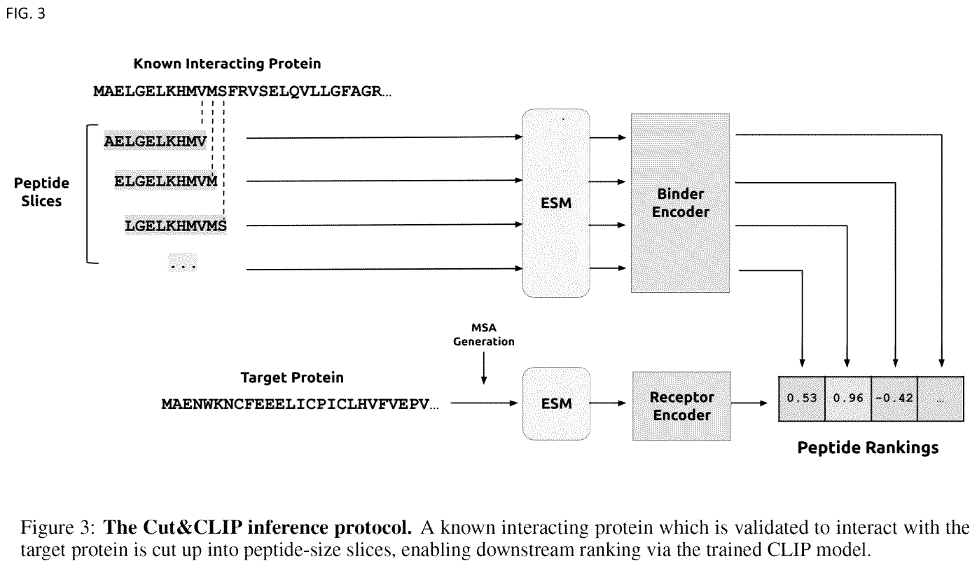

This peptide generation pipeline (referred to as Cut&CLIP inference protocol) is illustrated in FIG. 3. As shown in FIG. 3, the Cut&CLIP inference protocol is provided. Here, a known interacting protein which is validated to interact with the target protein is cut up into peptide-size slices, enabling downstream ranking via the trained CLIP model. For example, as shown in FIG. 3, a protein sequence known to interact with the target sequence is cut into slices. For example, an initial amino acid is selected from the known interacting sequence, as shown in step 702. In one implementation, the initial amino acid selected is the first, second, or third amino acid of a given protein sequence. However, it should be appreciated that any initial amino acid of the sequence can be selected to start the cutting process. Furthermore, it will be appreciated that more than one known interacting sequence can be selected for cutting into slicing.

Using the initial amino acid, a subsequence of the known interacting protein sequence is selected. For example, nine (9) amino acids downstream of the initial selected amino acid are selected for incorporation into a subsequence. This cutting or slicing process then proceeds to generate a second, or subsequent subsequence, by selecting the next amino acid that is downstream of the initial selected amino acid and capturing the next nine (9) amino acids in the protein sequence. While FIG. 3 illustrates a selection of 10 amino acids (the initial amino acid and nine (9) downstream amino acids), it will be appreciated that any number of downstream or upstream amino acids can be selected for a peptide slice.

Once the peptide have been generated, they are provided to the ESM model and in turn provided to the binder encoder. As used herein, the binder encoder a trained machine learning model (as described herein such as a neural network) that is used to convert input data into a latent representation.

The target protein is used in MSA generation. More specifically, generated MSAs are used as input to the ESM model to provide evolutionary context to each protein sequence. This allows the model to represent the protein in a more meaningful, biologically-relevant context. Once MSA has been provided as an input to the receptor encoder, the binder encoder and the receptor encoder are used to provide a peptide ranking of the peptide slices. For example, a processor of the system described is configured for computation of the CLIP peptide embedding for all k-mers of the interacting protein (where k is the desired size of the peptide), and rank them by their cosine similarities with the CLIP receptor embedding of the target protein. The closer the ranking is to +1.00, the greater the likelihood that the peptide binder slice will bind to the target protein sequence.

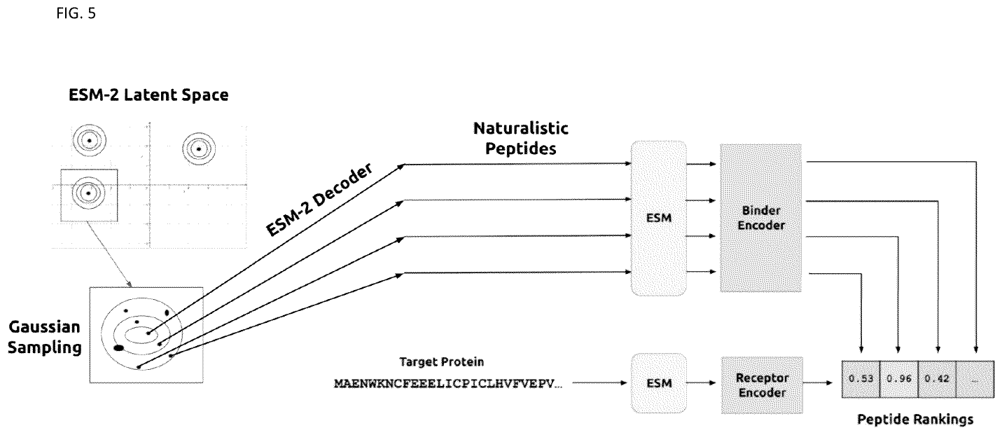

In an alternative implementation, all plausible de novo peptides are sampled from a large language model latent space and screen them through CLIP model for a new target sequence. This approach, as illustrated in FIG. 5 removes the need to generate the peptide slices as provided in Steps 808-810. Specifically, as shown in step 816-818, naturalistic peptide candidates are generated through a Gaussian sampling of the latent space of a protein language model. Here, latent space refers to latent space is a lower-dimensional representation of protein sequences. The latent space is learned by the protein language model from a large corpus of protein sequences. The latent space is typically represented as a high-dimensional vector space, where each dimension represents a latent feature of proteins. The latent features are typically extracted using a neural network architecture, such as a transformer or a recurrent neural network. For example, the current state-of-the-art protein language model, ESM-2 pLM, is used to provide potential peptide candidates without the need to generate peptide slices. However, it will be appreciated that alternative models, or combinations of protein language models could be used to the same effect.

In one particular arrangement, samples from Gaussian distributions centered around the ESM-2 embeddings of naturally-occurring peptides are decoded back to sequences. Since ESM-2's embedding space encodes expressive representations of protein sequences, the described generation method produces candidate peptides which are biochemically similar to naturally-occurring peptides.

Once the naturalistic peptide sequences have been generated, then can be provided to the trained model to generate rankings of the peptides. As noted with respect to steps 816-818, based on the output values of the models, those sequences that are ranked closest to +1.00 are selected as most likely to bind to the target sequence.

In one or more further implementations, a sequence synthesizer is used to automatically synthesize those sequences that are above a given ranking threshold. For example, where the ranking threshold is set at +0.45, all peptides that are ranked above this value are synthesized.

Using these synthesized peptides, it is possible to screen thousands of these peptides for binding activity to the target and prioritize the top candidates for experimental testing.

This strategy is, in some circumstances, sufficient to identify stand-alone peptide binders with high target affinity. However, the described approach can also be paired with the catalytic nature of E3 ubiquitin ligase activity, where selective target binding is sufficient to induce degradation. See Bekes et al., 2022, Buetow and Huang, 2016, and Portnoff et al., 2014.

### Target Protein Degradation with Cut&CLIP-Derived Peptides

Numerous previous works have attempted to redirect E3 ubiquitin ligases by replacing their natural protein binding domains with those targeting specific proteins. See Gosink and Vierstra, 1995, Zhou et al., 2000, Su et al., 2003. Recently, based on the seminal work of Portnoff, et al., the inventors have demonstrated the capability to reprogram the specificity of a modular human E3 ubiquitin ligase called CHIP (carboxyl-terminus of Hsc70-interacting protein) by replacing its natural substrate-binding domain, TPR, with designer peptides to generate minimal and programmable uAb architectures. See Portnoff et al., 2014, Chatterjee et al., 2020.

To evaluate Cut&CLIP's utility as compared to a less-efficient, structure-based method, such as AlphaFold. See Jumper et al., 2021, we selected three target proteins for experimental characterization: the spike receptor binding domain (RBD) of SARS-CoV-2, the TRIM8 E3 ubiquitin ligase, and the KRAS oncoprotein. Previously, we demonstrated robust degradation of RBD using peptide-based uAbs, and with stable co-crystal structures of RBD and the human ACE2 receptor, it represents a very tractable target for standard structure-based peptide generation. See Chatterjee et al., 2020, and Lan et al., 2020. TRIM8 regulates EWS-FLI protein degradation in Ewing sarcoma and its depletion results in EWS/FLI-mediated oncogene overdose, driving DNA damage and apoptosis of tumor cells. See Seong et al., 2021. Thus, as an E3 ubiquitin ligase itself, TRIM8 presents a unique target for therapeutic degradation. Finally, KRAS is the most frequently mutated oncoprotein, occurring in over 25% of all cancer patients. Due to its smooth and shallow surface, it is considered largely undruggable by standard small molecules, and its structure is evasive due to its conformational disorder as a transcription factor protein. See Huang et al., 2021.

### Performance Comparison With Existing Approaches

A search was conducted of existing PPI databases and the literature to identify putative interacting partners of three targets: ACE2 for RBD, PIAS3 for TRIM8, and RAF1 for KRAS. See Szklarczyk et al., 2020. These pairs were input into both the Cut&CLIP pipeline, as described herein, as well as a co-folding pipeline that adapts the AlphaFold-Multimer complex prediction algorithm followed by PeptiDerive (AF2-CoFold+PeptiDerive). SeeEvans et al., 2021, and Sedan et al., 2016. After candidate peptide derivation, experimentally cloned plasmids expressing eight peptides of variable lengths (≤18 amino acids) for each target were directly fused to the CHIPΔTPR uAb domain.

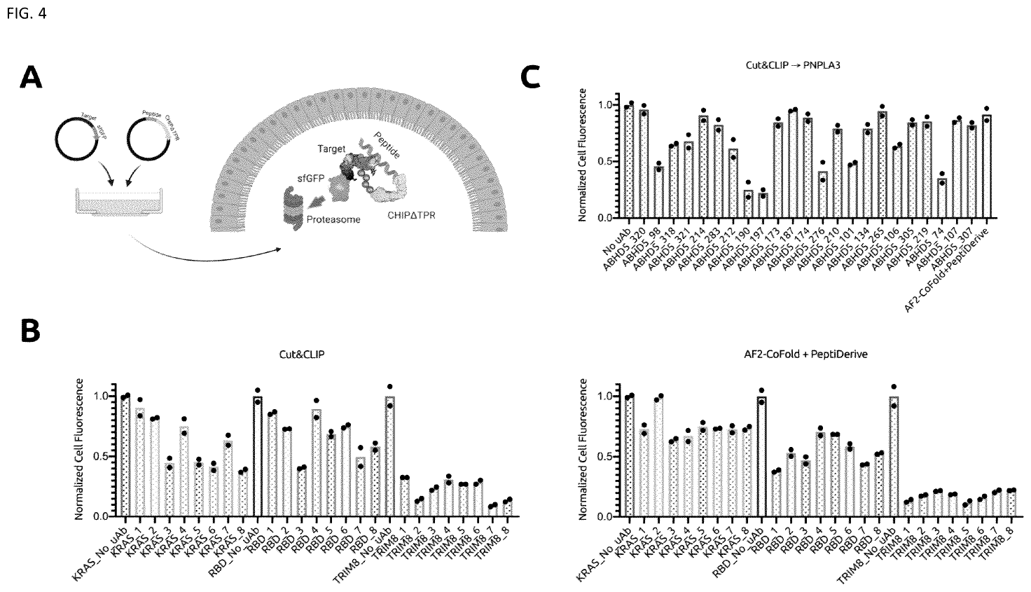

Subsequently these vectors were co-transfected into human HEK293T cells alongside plasmids expressing the target protein fused to superfolder green fluorescent protein (sfGFP) and analyzed the reduction of GFP+ signal (and thus target degradation) via flow cytometry. The results of which can be seen in FIG. 4A. The results demonstrate that select Cut&CLIP-derived peptides induce robust target degradation for all three targets, even on the “undruggable” KRAS oncoprotein. In comparison, the structure-based strategy, while successful on degrading RBD and TRIM8, fails to produce effective degraders for KRAS, as shown in FIG. 4B.

FIG. 4 provides for, uAbs are genetically-encoded constructs, their therapeutic application is limited by the need for in vivo delivery vehicles, most of which home to the liver, including lipid nanoparticles (LNPs). Hou et al., 2021. Thus, to extended Cut&CLIP's utility to a viable therapeutic target, in one arrangement, the described system, method and computer implement processes is used to design peptides to PNPLA3, a known driver of fatty liver disease, by employing its direct interacting protein, ABHD5 Yang et al., 2019. Post transfection and flow cytometry, show that the approached described herein (Cut&CLIP) identifies potent peptides that enable over 80% degradation of PNPLA3. As such, the described approaches have potential clinical translation, as shown in FIG. 4C.

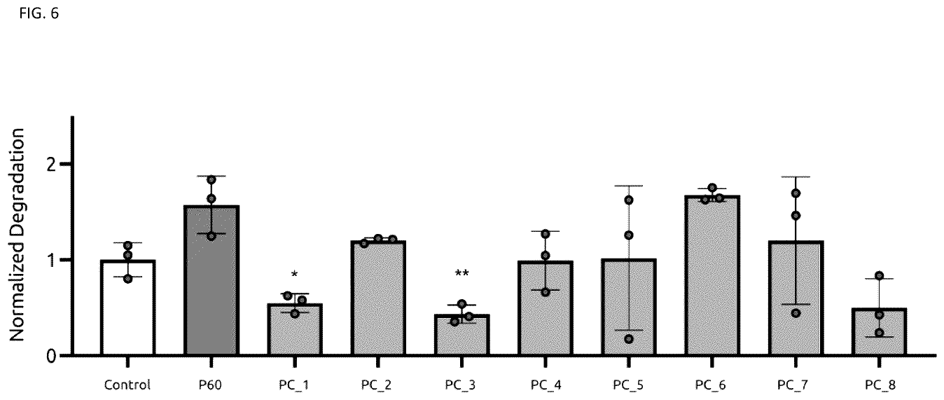

For experimental validation of the approach provided in steps 816-818, the described approach was used to design de novo peptides that bind to FOXP3, an undruggable transcription factor in T regulatory cells. Specifically, we aimed to reprogram the specificity of a modular human E3 ubiquitin ligase, CHIP, b replacing its natural substrate-binding domain, TPR, with designer peptides from PepPrCLIP. After candidate peptide design, we experimentally clonedplasmids expressing eight peptides of variable lengths (≤18 amino acids) directly fused to the CHIPΔTPRuAb domain via a short glycine-serine linker (GSGSG). We subsequently co-transfected these vectors into human HEK293T cells alongside plasmids expressing FOXP3 fused to superfolder green fluorescentprotein (sfGFP) and analyzed the reduction of GFP+ signal (and thus target degradation) via flowcytometry. Results show that two out of eight of the highest ranked generated peptides show statistically significant degradation of FOXP3, demonstrating the robust downselection capability of PepPrCLIP and motivating further functional validation, as shown in FIG. 6.

### Generation of Plasmids

In the foregoing example, pcDNA3-SARS-CoV-2-S-RBD-sfGFP (Addgene #141184) and pcDNA3-R4-uAb (Addgene #101800) and were obtained as gifts from Erik Procko and Matthew DeLisa, respectively. Target coding sequences (CDS) were synthesized as gBlocks from Integrated DNA Technologies (IDT). Sequences was amplified with overhangs for Gibson Assembly mediated insertion into the pcDNA3-SARS-CoV-2-S-RBD-Fc backbone linearized by digestion with Nhel and Xhol. An Esp31 restriction site was introduced immediately upstream of the CHIPΔTPR CDS and GSGSG linker via the KLD Enzyme Mix (NEB) following PCR amplification with mutagenic primers (Genewiz). For peptide CDS assembly, oligos were annealed and ligated via T4 DNA Ligase into the Esp3I-digested uAb backbone. Assembled constructs were transformed into 50 L NEB Turbo Competent Escherichia coli cells, and plated onto LB agar supplemented with the appropriate antibiotic for subsequent sequence verification of colonies and plasmid purification.

### Architecture and Mechanism of uAb Degradation System

CHIPΔTPR is fused to the C-terminus of targeting peptides, and can thus tag target-sfGFP complexes for ubiquitin mediated degradation in the proteasome, post-plasmid transfection. B) Analysis of KRAS-sfGFP, RBD-sfGFP, and TRIM8-sfGFP degradation via flow cytometry. All samples were performed in independent transfection duplicates (n=2) and gated on sfGFP+ fluorescence. Normalized cell fluorescence was calculated by dividing the % GFP+ of samples to that of their respective “No uAb” control. C) Analysis of PNPLA3-sfGFP degradation via flow cytometry. All samples were performed in independent transfection duplicates (n=2) and gated on sfGFP+ fluorescence. Normalized cell fluorescence was calculated by dividing the % GFP+ of samples to that of the “No uAb” control. The final peptide was derived from the CoFold+PeptiDerive strategy on PNPLA3-ABDH5. their therapeutic application is limited by the need for in vivo delivery vehicles, most of which home to the liver, including lipid nanoparticles (LNPs). See Hou et al., 2021. Thus, to extended Cut&CLIP's utility to a viable therapeutic target, we designed peptides to PNPLA3, a known driver of fatty liver disease, by employing its direct interacting protein, ABHD5.See Yang et al., 2019. Post transfection and flow cytometry, we show that Cut&CLIP identifies potent peptides that enable over 80% degradation of PNPLA3, thus motivating potential clinical translation of the approaches and technology described herein (FIG. 4C).

Curing malignancies is one of the greatest challenges for the future of human health, and protein-targeting therapeutics have served as potent solutions to this problem. As an example, targeted protein degradation with proteolysis targeting chimeras (PROTACs) and molecular glues enable small molecules to bind to intracellular proteins transiently and direct their proteolysis by recruiting E3 ubiquitin ligases. More recently, the development of the uAb technology has provided a modular, genetically-encoded alternative to achieve selective degradation of proteins deemed “undruggable” by standard small molecule-based means. In this work, we exploit recent advancements in contrastive deep learning to design peptides to specified target proteins. The final models accurately retrieve peptides for known protein-peptide pairs, and more importantly, prioritize candidates that demonstrate effective intracellular target degradation when integrated into the uAb architecture. The final Cut&CLIP model employs natural binding partners as scaffolds for peptide generation, thus representing a streamlined, efficient, sequence-based pipeline to generate degraders to diverse proteins in the proteome.

### Cell Culture and Flow Cytometry

HEK293T cells were maintained in Dulbecco's Modified Eagle's Medium (DMEM) supplemented with 100 units/ml penicillin, 100 mg/ml streptomycin, and 10% fetal bovine serum (FBS). Target-sfGFP (50 ng) and peptide-CHIPΔTPR were transfected into cells as duplicates (2×104/well in a 96-well plate) with Lipofectamine 3000 (Invitrogen) in Opti-MEM (Gibco). After 3 days post transfection, cells were harvested and analyzed on a FACSCelesta for GFP fluorescence (488-nm laser excitation, 530/30 filter for detection). Cells expressing sfGFP were gated, and normalized cell fluorescence was calculated to the “No uAb” control. Statistics and Reproducibility All samples were performed in independent transfection duplicates (n=2), and normalized cell fluorescence values were averaged.

### Methods of Treatment

In one or more implementations, a peptide-based therapeutic is provided where the therapeutic includes the polynucleotide of any developed using the Cut&CLIP method and process shown. In one further implementation, the peptide therapeutic includes any of the polynucleotides identified using the Cut&CLIP approaches described herein are coupled a delivery vector in which said delivery vector may be either a virus or micelle. Peptide-based therapeutic comprising the fusions of any of the foregoing polynucleotides identified using the Cut&CLIP approaches described herein in which said peptide fusion is further fused to a cell penetrating motif or a cell surface receptor binding motif. In certain embodiments, the compositions and methods of the present disclosure are useful for the prevention and/or treatment of symptoms of viral infection, cancer and metastasis. In certain embodiments, the compositions and methods of the present disclosure are useful for the prevention and/or treatment of viral infection, cancer and metastasis.

In one embodiment, the subject treated using polynucleotides identified using the Cut&CLIP approaches described herein has a cancer and metastasis. In some embodiments, the cancer or metastasis is selected from the group of basal cell carcinoma (BCC), head and neck squamous cell carcinoma (HNSCC), prostate cancer (CaP), pilomatrixoma (PTR) and medulloblastoma (MDB).

### Pharmaceutical Compositions

The present disclosure thus provides pharmaceutical compositions that include Peptide-E3 ubiquitin ligase fusion compounds and a pharmaceutically acceptable carrier derived through the use of the or PepPrCLIP or Cut&CLIP approaches described herein. The compounds of the present disclosure can be formulated as pharmaceutical compositions and administered to a mammalian host, such as a human patient, in a variety of forms adapted to the chosen route of administration.

Routes of administration include, but are not limited to oral, topical, mucosal, nasal, parenteral, gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic, transdermal, rectal, buccal, epidural and sublingual administration.

As used herein, the term “administering” generally refers to any and all means of introducing compounds described herein to the host subject. Compounds described herein may be administered in unit dosage forms and/or compositions containing one or more pharmaceutically-acceptable carriers, adjuvants, diluents, excipients, and/or vehicles, and combinations thereof.

As used herein, the terms “composition” generally refers to any product comprising more than one ingredient, including the compounds described herein. It is to be understood that the compositions described herein may be prepared from compounds described herein or from salts, solutions, hydrates, solvates, and other forms of the compounds described herein. It is appreciated that the compositions may be prepared from various amorphous, non-amorphous, partially crystalline, crystalline, and/or other morphological forms of the compounds described herein, and the compositions may be prepared from various hydrates and/or solvates of the compounds described herein. Accordingly, such pharmaceutical compositions that recite compounds described herein include each of, or any combination of, or individual forms of, the various morphological forms and/or solvate or hydrate forms of the compounds described herein.

In some embodiments, the Peptide-E3 ubiquitin ligase fusion based treatments may be systemically (e.g., orally) administered in combination with a pharmaceutically acceptable vehicle such as an inert diluent or an assimilable edible carrier. For oral therapeutic administration, the active compound may be combined with one or more excipients and used in the form of ingestible tablets, buccal tablets, sublingual tablets, troches, capsules, elixirs, suspensions, syrups, wafers, and the like. The percentage of the compositions and preparations may vary and may be between about 1 to about 99% weight of the active ingredient(s) and excipients such as, but not limited to a binder, a filler, a diluent, a disintegrating agent, a lubricant, a surfactant, a sweetening agent; a flavoring agent, a colorant, a buffering agent, anti-oxidants, a preservative, chelating agents (e.g., ethylenediaminetetraacetic acid), and agents for the adjustment of tonicity such as sodium chloride.

Suitable binders include, but are not limited to, polyvinylpyrrolidone, copovidone, hydroxypropyl methylcellulose, starch, and gelatin.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose.

Suitable diluents include, but are not limited to, dicalcium phosphate dihydrate, sugars, lactose, calcium phosphate, cellulose, kaolin, mannitol, sodium chloride, and dry starch.

Suitable disintegrants include, but are not limited to, pregelatinized starch, crospovidone, crosslinked sodium carboxymethyl cellulose and combinations thereof.

Suitable lubricants include, but are not limited to, sodium stearyl fumarate, stearic acid, polyethylene glycol or stearates, such as magnesium stearate.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil.

Suitable flavoring agents and sweeteners include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Suitable buffering or pH adjusting agent include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide.

Suitable tonicity enhancing agents include, but are not limited to, ionic and non-ionic agents such as, alkali metal or alkaline earth metal halides, urea, glycerol, sorbitol, mannitol, propylene glycol, and dextrose.

Suitable wetting agents include, but are not limited to, glycerin, cetyl alcohol, and glycerol monostearate.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Suitable antioxidants include, but are not limited to, sorbic acid, ascorbic acid, ascorbate, glycine, α-tocopherol, butylated hydroxyanisole (BHA), and butylated hydroxytoluene (BHT).

The Peptide-E3 ubiquitin ligase fusion based treatments of the present disclosure may also be administered via infusion or injection (e.g., using needle (including microneedle) injectors and/or needle-free injectors). Solutions of the active composition can be aqueous, optionally mixed with a nontoxic surfactant and/or may contain carriers or excipients such as salts, carbohydrates and buffering agents (preferably at a pH of from 3 to 9), and, for some applications, they may be more suitably formulated as a sterile non-aqueous solution or as a dried form to be used in conjunction with a suitable vehicle such as sterile, pyrogen-free water or phosphate-buffered saline. For example, dispersions can be prepared in glycerol, liquid polyethylene glycols, triacetin, and mixtures thereof and in oils. The preparations may further contain a preservative to prevent the growth of microorganisms.

The pharmaceutical compositions may be formulated for parenteral administration (e.g., subcutaneous, intravenous, intra-arterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Water is a preferred carrier when the pharmaceutical composition is administered intravenously. Saline solutions and aqueous dextrose and glycerol solutions can also be employed as liquid carriers, particularly for injectable solutions. Oils such as petroleum, animal, vegetable, or synthetic oils and soaps such as fatty alkali metal, ammonium, and triethanolamine salts, and suitable detergents may also be used for parenteral administration. Further, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Suitable preservatives include e.g. sodium benzoate, benzoic acid, and sorbic acid. Suitable antioxidants include e.g. sulfites, ascorbic acid and-tocopherol.

The preparation of parenteral compounds/compositions under sterile conditions, for example, by lyophilization, may readily be accomplished using standard pharmaceutical techniques well known to those skilled in the art.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. In one embodiment, the compositions are administered by the oral or nasal respiratory route for local or systemic effect. Compositions in pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, orally or nasally, from devices that deliver the formulation in an appropriate manner.

In yet another embodiment, the composition is prepared for topical administration, e.g. as an ointment, a gel, a drop or a cream. For topical administration to body surfaces using, for example, creams, gels, drops, ointments and the like, the compounds of the present disclosure can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, pumps delivering the drugs into the body (including mechanical or osmotic pumps) controlled-release formulations and the like, as are known in the art.

### Doses

As used herein, the term “therapeutically effective dose” means (unless specifically stated otherwise) a quantity of a compound which, when administered either one time or over the course of a treatment cycle affects the health, wellbeing or mortality of a subject.

A Peptide-E3 ubiquitin ligase fusion based treatment described herein can be present in a composition in an amount of about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5g, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

A Peptide-E3 ubiquitin ligase fusion based treatment described herein described herein can be present in a composition in a range of from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5 mg to about 5 mg, from about 0.5 mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

### Dosing Regimens

The compounds described herein can be administered by any dosing schedule or dosing regimen as applicable to the patient and/or the condition being treated. Administration can be once a day (q.d.), twice a day (b.i.d.), thrice a day (t.i.d.), once a week, twice a week, three times a week, once every 2 weeks, once every three weeks, or once a month twice, and the like.

In some embodiments, the Peptide-E3 ubiquitin ligase fusion based treatment is administered for a period of at least one day. In other embodiments, the Peptide-E3 ubiquitin ligase fusion based treatment is administered for a period of at least 2 days. In other embodiments, the Peptide-E3 ubiquitin ligase fusion based treatment is administered for a period of at least 3 days. In other embodiments, the Peptide-E3 ubiquitin ligase fusion based treatment is administered for a period of at least 4 days. In other embodiments, the Peptide-E3 ubiquitin ligase fusion based treatment is administered for a period of at least 5 days. In other embodiments, the Peptide-E3 ubiquitin ligase fusion based treatment is administered for a period of at least 6 days. In other embodiments, the Peptide-E3 ubiquitin ligase fusion based treatment is administered for a period of at least 7 days. In other embodiments, the Peptide-E3 ubiquitin ligase fusion based treatment is administered for a period of at least 10 days. In other embodiments, the Peptide-E3 ubiquitin ligase fusion based treatment is administered for a period of at least 14 days. In other embodiments, the Peptide-E3 ubiquitin ligase fusion based treatment is administered for a period of at least one month. In some embodiments, the Peptide-E3 ubiquitin ligase fusion based treatment is administered chronically for as long as the treatment is needed.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

Each of the following references are herein incorporated by reference as if presented in their respective entireties: Adhikari et al., 2018 Adhikari, S., Alahmadi, T. I., Gong, Z., and Karlsson, A. J. (2018). Expression of cell-penetrating peptides fused to protein cargo. Journal of Molecular Microbiology and Biotechnology, 28(4):159-168. Anishchenko et al., 2021 Anishchenko, I., Pellock, S. J., Chidyausiku, T. M., Ramelot, T. A., Ovchinnikov, S., Hao, J., Bafna, K., Norn, C., Kang, A., Bera, A. K., DiMaio, F., Carter, L., Chow, C. M., Montelione, G. T., and Baker, D. (2021). De novo protein design by deep network hallucination. Nature, 600(7889):547-552.Békés et al., 2022 Békés, M., Langley, D. R., and Crews, C. M. (2022). PROTAC targeted protein degraders: the past is prologue. Nature Reviews Drug Discovery, 21(3):181-200. Buchwald et al., 2014 Buchwald, H., Dorman, R. B., Rasmus, N. F., Michalek, V. N., Landvik, N. M., and Ikramuddin, S. (2014). Effects on GLP-1, PYY, and leptin by direct stimulation of terminal ileum and cecum in humans: implications for ileal transposition. Surgery for Obesity and Related Diseases, 10(5):780-786.Buetow and Huang, 2016 Buetow, L. and Huang, D. T. (2016). Structural insights into the catalysis and regulation of e3 ubiquitin ligases. Nature Reviews Molecular Cell Biology, 17(10):626-642. Cao et al., 2022 Cao, L., Coventry, B., Goreshnik, I., Huang, B., Sheffler, W., Park, J. S., Jude, K. M., Markovi c, I., Kadam, R. U., Verschueren, K. H. G., Verstraete, K., Walsh, S. T. R., Bennett, N., Phal, A., Yang, A., Kozodoy, L.,DeWitt, M., Picton, L., Miller, L., Strauch, E.-M., DeBouver, N. D., Pires, A., Bera, A. K., Halabiya, S., Hammerson, B., Yang, W., Bernard, S., Stewart, L., Wilson, I. A., Ruohola-Baker, H., Schlessinger, J., Lee, S., Savvides, S. N., Garcia, K. C., and Baker, D. (2022). Design of protein-binding proteins from the target structure alone. Nature, 605(7910):551-560. Carle et al., 2021 Carle, V., Kong, X.-D., Comberlato, A., Edwards, C., Díaz-Perlas, C., and Heinis, C. (2021). Generation of a 100-billion cyclic peptide phage display library having a high skeletal diversity. Protein Engineering, Design and Selection, 34. Chatterjee et al., 2020 Chatterjee, P., Ponnapati, M., Kramme, C., Plesa, A. M., Church, G. M., and Jacobson, J. M. (2020). Targeted intracellular degradation of SARS-CoV-2 via computationally optimized peptide fusions. Communications Biology, 3(1). Das et al., 2018 Das, P., Matysiak, S., and Mittal, J. (2018). Looking at the disordered proteins through the computational microscope. ACS Central Science, 4(5):534-542.Elnaggar et al., 2020 Elnaggar, A., Heinzinger, M., Dallago, C., Rehawi, G., Wang, Y., Jones, L., Gibbs, T., Feher, T., Angerer, C., Steinegger, M., Bhowmik, D., and Rost, B. (2020). ProtTrans: Towards cracking the language of life'scode through self-supervised learning. Evans et al., 2021 Evans, R., O'Neill, M., Pritzel, A., Antropova, N., Senior, A., Green, T., Žídek, A., Bates, R., Blackwell, S., Yim, J., Ronneberger, O., Bodenstein, S., Zielinski, M., Bridgland, A., Potapenko, A., Cowie, A., Tunyasuvunakool, K., Jain, R., Clancy, E., Kohli, P., Jumper, J., and Hassabis, D. (2021). Protein complex predictionwith AlphaFold-multimer. Fosgerau and Hoffmann, 2015 Fosgerau, K. and Hoffmann, T. (2015). Peptide therapeutics: current status and futuredirections. Drug Discovery Today, 20(1):122-128.Gosink and Vierstra, 1995 Gosink, M. M. and Vierstra, R. D. (1995). Redirecting the specificity of ubiquitination bymodifying ubiquitin-conjugating enzymes. Proceedings of the National Academy of Sciences, 92(20):9117-9121.Hinton et al., 2015 Hinton, G., Vinyals, O., and Dean, J. (2015). Distilling the knowledge in a neural network. Hou et al., 2021 Hou, X., Zaks, T., Langer, R., and Dong, Y. (2021). Lipid nanoparticles for mRNA delivery. NatureReviews Materials, 6(12):1078-1094.Huang et al., 2021 Huang, L., Guo, Z., Wang, F., and Fu, L. (2021). KRAS mutation: from undruggable to druggablein cancer. Signal Transduction and Targeted Therapy, 6(1). Johnson et al., 2021 Johnson, K. L., Qi, Z., Yan, Z., Wen, X., Nguyen, T. C., Zaleta-Rivera, K., Chen, C.-J., Fan, X.,Sriram, K., Wan, X., Chen, Z. B., and Zhong, S. (2021). Revealing protein-protein interactions at the transcriptomescale by sequencing. Molecular Cell, 81(19):4091-4103.e9. Jumper et al., 2021 Jumper, J., Evans, R., Pritzel, A., Green, T., Figurnov, M., Ronneberger, O., Tunyasuvunakool, K., Bates, R., Žídek, A., Potapenko, A., Bridgland, A., Meyer, C., Kohl, S. A. A., Ballard, A. J., Cowie, A., Romera-Paredes, B., Nikolov, S., Jain, R., Adler, J., Back, T., Petersen, S., Reiman, D., Clancy, E., Zielinski, M.,Steinegger, M., Pacholska, M., Berghammer, T., Bodenstein, S., Silver, D., Vinyals, O., Senior, A. W., Kavukcuoglu, K., Kohli, P., and Hassabis, D. (2021). Highly accurate protein structure prediction with AlphaFold.Nature, 596(7873):583-589. Kong et al., 2020 Kong, X.-D., Carle, V., Díaz-Perlas, C., Butler, K., and Heinis, C. (2020). Generation of a largepeptide phage display library by self-ligation of whole-plasmid PCR product. ACS Chemical Biology, 15(11):2907-2915. Lan et al., 2020 Lan, J., Ge, J., Yu, J., Shan, S., Zhou, H., Fan, S., Zhang, Q., Shi, X., Wang, Q., Zhang, L., andWang, X. (2020). Structure of the SARS-CoV-2 spike receptor-binding domain bound to the ACE2receptor. Nature, 581(7807):215-220. Lindgren et al., 2000 Lindgren, M., Hällbrink, M., Prochiantz, A., and Ulo Langel (2000). Cell-penetrating peptides. Trends in Pharmacological Sciences, 21(3):99-103. Lozano et al., 2017 Lozano, T., Gorraiz, M., Lasarte-Cía, A., Ruiz, M., Rabal, O., Oyarzabal, J., Hervás-Stubbs, S., Llopiz, D., Sarobe, P., Prieto, J., Casares, N., and Lasarte, J. J. (2017). Blockage of FOXP3transcription factordimerization and FOXP3/AML1 interaction inhibits t regulatory cell activity: sequence optimization of a peptideinhibitor. Oncotarget, 8(42):71709-71724. Madani et al., 2021 Madani, A., Krause, B., Greene, E. R., Subramanian, S., Mohr, B. P., Holton, J. M., Olmos, J. L., Xiong, C., Sun, Z. Z., Socher, R., Fraser, J. S., and Naik, N. (2021). Deep neural language modeling enables functional protein generation across families. Martins et al., Martins, P. M., Santos, L. H., Mariano, D., Queiroz, F. C., Bastos, L. L., Gomes, I. d. S., Fischer, P. H. C., Rocha, R. E. O., Silveira, S. A., de Lima, L. H. F., de Magalhães, M. T. Q., Oliveira, M. G. A., and de Melo-Minardi, R. C. Propedia: a database for protein-peptide identification based on a hybrid clustering algorithm. 22(1):1. Padhi et al., 2014 Padhi, A., Sengupta, M., Sengupta, S., Roehm, K. H., and Sonawane, A. (2014). Antimicrobial peptides and proteins in mycobacterial therapy: Current status and future prospects. Tuberculosis, 94(4):363-373. Peterson et al., 2017 Peterson, L. X., Roy, A., Christoffer, C., Terashi, G., and Kihara, D. (2017). Modeling disordered protein interactions from biophysical principles. PLOS Computational Biology, 13(4):e1005485. Portnoff et al., 2014 Portnoff, A. D., Stephens, E. A., Varner, J. D., and DeLisa, M. P. (2014). Ubiquibodies, synthetice3 ubiquitin ligases endowed with unnatural substrate specificity for targeted protein silencing. Journal of Biological Chemistry, 289(11):7844-7855. Radford et al., 2021 Radford, A., Kim, J. W., Hallacy, C., Ramesh, A., Goh, G., Agarwal, S., Sastry, G., Askell, A., Mishkin, P., Clark, J., Krueger, G., and Sutskever, I. (2021). Learning transferable visual models from natural language supervision. Rao et al., 2021 Rao, R., Liu, J., Verkuil, R., Meier, J., Canny, J. F., Abbeel, P., Sercu, T., and Rives, A. (2021). MSA transformer. Rao et al., 2020 Rao, R. M., Meier, J., Sercu, T., Ovchinnikov, S., and Rives, A. (2020). Transformer protein language models are unsupervised structure learners. Raveh et al., 2011 Raveh, B., London, N., Zimmerman, L., and Schueler-Furman, O. (2011). Rosetta FlexPepDockab-initio: Simultaneous folding, docking and refinement of peptides onto their receptors. PLOS ONE, 6(4):e18934.Rives et al., 2021 Rives, A., Meier, J., Sercu, T., Goyal, S., Lin, Z., Liu, J., Guo, D., Ott, M., Zitnick, C. L., Ma, J., and Fergus, R. (2021). Biological structure and function emerge from scaling unsupervised learning to 250 million protein sequences. Proceedings of the National Academy of Sciences, 118(15):e2016239118. Sedan et al., 2016 Sedan, Y., Marcu, O., Lyskov, S., and Schueler-Furman, O. (2016). Peptiderive server: derivepeptide inhibitors from protein-protein interactions. Nucleic Acids Research, 44(W1):W536-W541. Seong et al., 2021 Seong, B. K. A., Dharia, N. V., Lin, S., Donovan, K. A., Chong, S., Robichaud, A., Conway, A., Hamze, A., Ross, L., Alexe, G., Adane, B., Nabet, B., Ferguson, F. M., Stolte, B., Wang, E. J., Sun, J., Darzacq, X., Piccioni, F., Gray, N. S., Fischer, E. S., and Stegmaier, K. (2021). TRIM8 modulates the EWS/FLI oncoprotein to promote survival in ewing sarcoma. Cancer Cell, 39(9):1262-1278.e7. Shin et al., 2020 Shin, W.-H., Kumazawa, K., Imai, K., Hirokawa, T., and Kihara, D. (2020). pcurrent challenges and opportunities in designing protein-protein interaction targeted drugs/p. Advances and Applications in Bioinformatics and Chemistry, Volume 13:11-25. Slastnikova et al., 2018 Slastnikova, T. A., Ulasov, A. V., Rosenkranz, A. A., and Sobolev, A. S. (2018). Targeted intracellular delivery of antibodies: The state of the art. Frontiers in Pharmacology, 9. Steinegger and Soding, Steinegger, M. and Soding, J. Clustering huge protein sequence sets in linear time. 9(1):2542. Number: 1 Publisher: Nature Publishing Group. Su et al., 2003 Su, Y., Ishikawa, S., Kojima, M., and Liu, B. (2003). Eradication of pathogenic-catenin byskp1/cullin/f box ubiquitination machinery. Proceedings of the National Academy of Sciences, 100(22):12729-12734. Szklarczyk et al., 2020 Szklarczyk, D., Gable, A. L., Nastou, K. C., Lyon, D., Kirsch, R., Pyysalo, S., Doncheva, N. T., Legeay, M., Fang, T., Bork, P., Jensen, L. J., and von Mering, C. (2020). The STRING database in 2021: customizable protein-protein networks, and functional characterization of user-uploaded gene/measurement sets. Nucleic Acids Research, 49(D1):D605-D612. Townshend et al., 2018 Townshend, R. J. L., Bedi, R., Suriana, P. A., and Dror, R. O. (2018). End-to-end learning on3d protein structure for interface prediction. Tsaban et al., 2022 Tsaban, T., Varga, J. K., Avraham, O., Ben-Aharon, Z., Khramushin, A., and Schueler-Furman, O. (2022). Harnessing protein folding neural networks for peptide-protein docking. Nature Communications, 13(1). Vig et al., 2020 Vig, J., Madani, A., Varshney, L. R., Xiong, C., Socher, R., and Rajani, N. F. (2020). BERTologymeets biology: Interpreting attention in protein language models. Wu et al., 2016 Wu, C.-H., Liu, I.-J., Lu, R.-M., and Wu, H.-C. (2016). Advancement and applications of peptide phage display technology in biomedical science. Journal of Biomedical Science, 23(1). Yang et al., 2019 Yang, A., Mottillo, E. P., Mladenovic-Lucas, L., Zhou, L., and Granneman, J. G. (2019). Dynamic interactions of ABHD5 with PNPLA3 regulate triacylglycerol metabolism in brown adipocytes. Nature Metabolism, 1(5):560-569. Zhou et al., 2000 Zhou, P., Bogacki, R., McReynolds, L., and Howley, P. M. (2000). Harnessing the ubiquitination machinery to target the degradation of specific cellular proteins. Molecular Cell, 6(3):751-756.

1. A process for identifying binding peptides using a trained machine learning model, the process comprising:
(1) training a machine learning model to identify corresponding peptides to a target protein using a zero-shot transfer and multimodal learning algorithm; wherein the learning algorithm has jointly trained receptor and peptide encoders; and
(2) providing a target protein to the trained machine learning model as an input and receiving from the trained machine learning model at least one corresponding binding peptide.

2. The process of claim 1, wherein the training of the machine learning model includes jointly training peptide and receptor encoders on ESM embeddings to predict high cosine similarities between known peptide-receptor embedding pairs and low cosine similarities for all other pairs.

3. The process of claim 1, wherein the training of the machine learning model includes providing as an input to the receptor encoder a multiple sequence alignment (MSA).

4. The process of claim 1, wherein the training of the machine learning model includes providing as an input to the peptide encoder a peptide sequence.

5. A system for generating a peptide sequence configured to bind to a target protein sequence, the system comprising:
A processor, configured by code executing therein to:
Sample, from Gaussian distributions centered around embeddings of naturally occurring peptides in a protein language model, sampling embeddings;
Decode the sampling embeddings into decoded protein sequences;
Receive as an input, a target protein sequence, and
Provide the target protein sequence to a pretrained contrastive language model, where in the contrastive language model is trained to generate, upon receipt of the target sequence, ranking values that correspond to the likelihood that one or more of the decoded sequences bind to the target sequence.

6. The system of claim 5, wherein the ranking values range from −1.00 to +1.00, where the closer a ranking value is to +1.00, the higher the likelihood that the corresponding decoded protein sequence will bind with the target sequence.

7. A system for generating a peptide sequence configured to bind to a target protein sequence, the system comprising:
a processor, configured by code executing therein to:
receive a target protein sequence;
obtain, from one or more databases, one or more known interacting partners to the target protein sequence;
generate, for each of the one or more known interacting partners, subsequences having a sequence length shorter than the known interacting partner sequence length; and
provide the generated subsequences and the target sequence to a pretrained contrastive language model, wherein the contrastive language model is trained to generate, upon receipt of the target sequence, ranking values that correspond to the likelihood that one or more of the subsequences bind to the target sequence.

8. The system of claim 7, wherein the ranking values range from −1.00 to +1.00, where the closer a ranking value is to +1.00, the higher the likelihood that the corresponding decoded protein sequence will bind with the target sequence.

9. (canceled)

10. The system of claim 7, wherein the contrastive language model is a zero-shot transfer and multimodal learning algorithm.

11. The generating a peptide sequence of claim 5, further comprising, outputting at least one of the decoded protein sequences.
